SEQUENCE LISTING (11 sequences)

SEQ ID NO: 1 - DNA, 756 bp, Bacillus subtilis DSM 1088
atgcaagacatctacggaactttagccaatctgaacacgaaattaaaacaaaagctgtctcatccttatttagcgaagcatatttctgcgccgaaaattgatgaggataagcttcttctttttcatgctttatttgaagaagctgacataaaaaacaacgacagagaaaattatattgtaacagcgatgcttgtacaaagcgcccttgatacccatgatgaagtgacgacagctagagtcataaaacgagacgaaaacaaaaaccgccaattgactgttctcgcgggcgattatttcagcgggctgtactactctttactatctgaaatgaaggatatctacatgattcggacgcttgctacagccattaaagaaatcaacgaacataaaattcgtctgtatgaccgttctttcaaggacgaaaacgattttttcgaaagtgtcggcatcgttgaatcagctttattccatcgtgtggcggaacacttcagcctcccgcgctggaaaaagctgtcgagtgatttttttgtatttaagcggcttatgaacggaaatgatgcatttctggatgtgatcggcagttttatacagctgggaaaaacaaaagaagagatattagaagattgttttaaaaaagcgaaaaacagcattgagtcacttctgcctctaaattcacctattcagaacattttaataaaccgtctgaagacaatcagccaagatcaaacctatcatcagaaagtggaagaagggtaa

SEQ ID NO: 2 - DNA, 1047 bp, Bacillus subtilis DSM 1088
atgttaaatatcattcgtttactggcggagtcgctgccacgcatatcggatggaaatgaaaacacagatgtttgggtgaatgatatgaaatttaaaatggcctactcttttttaaatgacgatattgatgtaatcgaaagagaacttgaacaaaccgtacgttccgattacccgcttttaagcgaggcaggtcttcacctgctgcaggccggagggaaacgtattcgtcctgttttcgtgctgctttctggcatgtttggcgattacgatattaataagattaaatatgtcgccgtcactctggaaatgattcacatggcatctttggttcatgatgatgtcattgatgatgcagagcttcgccgaggaaaaccgacaatcaaagcaaagtgggacaatcgtattgcgatgtacacaggcgattatatgcttgcgggatctcttgaaatgatgacgagaattaacgaaccgaaagcccataggattttgtcacagacgatcgttgaagtttgtctaggggaaattgagcagatcaaagacaaatataacatggaacaaaatctcagaacgtatctccgccgtatcaaaagaaaaacagctctcttgatcgcggtcagctgccagcttggtgccattgcgtctggagctgatgagaagattcataaggcattgtactggtttgggtattacgtcggcatgtcttatcagattattgatgatattcttgattttacttcaactgaagaagagctgggtaaacccgttggaggagatttgcttcaaggaaacgtcacattgccagtgctgtatgccctgaaaaatcctgcattaaaaaaccagcttaaattgattaacagtgagacaacgcaggagcagcttgaaccaatcattgaagaaatcaaaaaaacagatgcaattgaagcatctatggcagtaagcgaaatgtatctgcagaaagcttttcagaaattaaacacgcttccacgagggcgcgcacgctcgtctcttgcagccatcgcaaaatatatcggtaaaagaaaattttaa

SEQ ID NO: 3 - DNA, 936 bp, Bacillus subtilis DSM 1088
atgaaccaaacaaataagggtgagggtcagacagcgccgcaaaaagaaagcatggggcagatcctttggcagttaacccgtcctcatacgttaaccgcatcgtttgtgcctgtgctgctcggaaccgttttggcgatgttttatgtgaaggttgatctgctgctgtttttggctatgctgttttcttgcctatggatccagatcgcgacgaacttatttaatgaatattatgattttaaacgcggattagatacagcagaatcagtcggaatcggaggggcaattgtacgccacggaatgaagcctaaaacgattttgcaattagctctggcatcatacgggattgccattttgctcggtgtctatatttgtgcgagcagcagctggtggcttgcgctgatcggccttgtcggcatggcgatcggctacctgtatacaggcgggccgctgccgattgcgtacacgccgttcggtgaattattctcaggcatttgcatgggttcggtgtttgtgctgatttcgtttttcattcagacagatatgatcaacacgcaaagcattttgatttccatcccgattgcgattcttgtcggcgcgattaatttgtcaaacaacattcgcgatattgaagaggacaaaaaaggcggccgcaaaacattggcgattttgatggggcataagggagctgttactctgttagctgcgtcgtttgccgtcgcttatatctgggttgtcggcttggttattaccggtgccgcaagcccatggctgtttgtcgtctttttgagcgtgcctaagccggttcaggcagtgaagggcttcgtccaaaaagaaatgccgatgaatatgattgtcgcaatgaaatcaacagcccaaacaaatacatttttcggattcctgctttcgatcggattattgatcagctatttccgataa

SEQ ID NO: 4 - DNA, 39 bp, Artificial Sequence
  Oligonucleotide
aaagggcccaggaggtagaatgcaagacatctacggaac

SEQ ID NO: 5 - DNA, 29 bp, Artificial Sequence
  Oligonucleotide
ccccttaagttacccttcttccactttct

SEQ ID NO: 6 - DNA, 39 bp, Artificial Sequence
  Oligonucleotide
aaacttaagaggaggtagaatgttaaatatcattcgttt

SEQ ID NO: 7 - DNA, 29 bp, Artificial Sequence
  Oligonucleotide
cccagatctttaaaattttcttttaccga

SEQ ID NO: 8 - DNA, 39 bp, Artificial Sequence
  Oligonucleotide
aaaagatctaggaggtagaatgaaccaaacaaataaggg

SEQ ID NO: 9 - DNA, 31 bp, Artificial Sequence
  Oligonucleotide
cccttaattaattatcggaaatagctgatca

SEQ ID NO: 10 - DNA, 35 bp, Artificial Sequence
  Oligonucleotide
aaagagctcgaattcgcatacgcgtgaggcgagtc

SEQ ID NO: 11 - DNA, 29 bp, Artificial Sequence
  Oligonucleotide
aaagggcccctgaatttcatcttttgtaa

(19) United States
(12) Patent Application Publication
(10) Pub. No.: US 2011/0195467 A1
(43) Pub. Date: Aug. 11, 2011

(54) METHOD FOR FERMENTATIVE PRODUCTION OF MENAQUINONE-7 USING ESCHERICHIA COLI

(75) Inventor: Thomas Schloesser, Burghausen (DE)

(73) Assignee: WACKER CHEMIE AG, Muenchen (DE)

(21) Appl. No.: 13/022,791

(22) Filed: Feb. 8, 2011

(30) Foreign Application Priority Data
Feb. 11, 2010 (DE) 10 2010 001 832.5

(51) Int. Cl. C12P 7/66; C12N 15/70; C12N 1/21

(52) U.S. Cl. 435/133; 435/320.1; 435/252.33

(57) ABSTRACT

The invention relates to a method of producing menaquinone-7, characterized in that cells of an E. coli strain comprising the B. subtilis DSM 1088 hepS gene, the B. subtilis DSM 1088 hepT gene and the putative B. subtilis DSM 1088 heptaprenyl transferase gene are fermented in a fermentation medium, with menaquinone-7 being accumulated in the cells of the fermented E. coli strain.

### BACKGROUND OF THE INVENTION

The invention relates to a method for fermentative production of menaquinone-7 by means of Escherichia coli.

Menaquinone-7 is part of the vitamin K family. The main source of menaquinones in human nutrition are gut-dwelling bacteria, such as, for example, Lactobacillus acidophilus or Escherichia coli. Biochemically, menaquinones are involved in the carboxylation of specific glutamic acid residues to give γ-carboxy-glutamic acid (GLA). A prominent protein containing 3 GLA residues is osteocalcin. Osteocalcin is formed in the osteoblasts and constitutes 15-20% of non-collagen proteins in the bone. Vitamin K deficiency results in non-carboxylated osteocalcin which enters the plasma and is an important indicator of metabolic disorders of the bone.

Studies indicate that menaquinone-7 supplements have both beneficial effects on bone formation and a preventive action with regard to arteriosclerosis in humans.

One study, for example, shows a reduced vitamin K supply to be associated with reduced bone density and an increased risk of hip fractures (Feskanich et al., 1999, Am. J. Clin. Nutr. 69: 74-79).

Another study demonstrates that supplementing the diet with menaquinone-7 results in improved γ-carboxylation of osteocalcin (Tsukamoto, 2004, BioFactors 22: 5-19).

Traditionally, menaquinone-7 is produced using a Bacillus subtilis strain which by nature has a relatively high menaquinone-7 content. This strain is employed in Asian regions for producing natto and is therefore also referred to as Bacillus subtilis natto or Bacillus natto (Earl et al., 2007, J. Bacteriol. 189: 1163-1170). One possibility of producing menaquinone-7 is therefore to ferment soybeans with the aid of Bacillus subtilis natto and then isolate menaquinone-7 from the natto. EP1803820B1 describes the industrial fermentation of a menaquinone-7-producing Bacillus subtilis strain as an alternative production option. Said strain may subsequently be spray-dried directly and sold by way of menaquinone-7-containing biomass.

Owing to the preventive properties with regard to osteoporosis and arteriosclerosis, there is an increase in foodstuffs containing vitamin K. This additive can be added to the food, as has been described for menaquinone-4, for example, or foodstuffs can directly contain menaquinone-producing lactic acid bacteria (EP1153548B1 and EP2076585). Lactic acid bacteria such as Lactococcus lactis (subspecies cremoris or lactis) or Leoconostoc lactis produce mainly menaquinone-8 and menaquinone-9 (Morishita et al., 1999, J. Dairy Sci. 82: 1897-1903).

Bacteria generally produce menaquinones with isoprenoid chains of different lengths and may partly also be distinguished taxonomically by this metabolite. Bacillus subtilis, as described further above, produces mainly menaquinone-7.

In contrast to Bacillus subtilis, the main menaquinones found in Escherichia coli are menaquinone-8 and menaquinone-9 (Collins and Jones, 1981, Microbiological Reviews 45: 316-354). All of the genes of the proteins involved in menaquinone biosynthesis in Escherichia coli are known. Proceeding from the starting metabolites chorismate, an intermediate of tryptophan biosynthesis, and isoprenyl pyrophosphate (IPP), a central metabolite of isoprenoid biosynthesis, initially the intermediate 1,4-dihydroxy-2-naphthoic acid (DHNA) is formed. This involves the enzymes MenF (isochorismate synthase), MenD (2-succinyl-6-hydroxy-2,4-cyclohexadiene-1-carboxylate synthase), MenC(O-succinylbenzoate synthase), MenE (O-succinylbenzoate-CoA ligase) and MenB (naphthoate-CoA synthase). DNHA prenyltransferase, which is encoded by MenA, transfers an octaprenyl pyrophosphate unit to DHNA, liberating CO₂ and pyrophosphate. Demethyl menaquinone (DMK) is produced. The last biosynthetic step comprises DMK being methylated by the S-adenosylmethionine-dependent methyl transferase, UbiE, thereby producing menaquinone-8. Octaprenyl pyrophosphate is synthesized from IPP with the aid of three enzymes encoded by the genes idi, ispA and ispB. In a first synthesis step, IPP is isomerized by isopentenyl diphosphate isomerase, Idi, and then elongated in two IspA-catalysed reaction steps to give a C15 unit. In a further four reaction steps, all of which are catalysed by the IspB octaprenyl synthase, the C40 unit, octaprenyl pyrophosphate, is synthesized.

The Bacillus subtilis bacterium carries out a comparable menaquinone biosynthesis. However, heptaprenyl transferase, a key enzyme, which transfers the heptaprenyl unit to DHNA, has been neither genetically identified nor biochemically described. However, genetic accessibility of the menaquinone-7 biosynthesis genes is essential for a large-scale industrial process for producing menaquinone-7.

It is therefore an object of the present invention to provide a method which enables menaquinone-7 to be produced by using Escherichia coli.

### SUMMARY OF THE INVENTION

This object is achieved by a method which is characterized in that cells of an E. coli strain comprising the B. subtilis DSM 1088 hepS gene, the B. subtilis DSM 1088 hepT gene and a putative B. subtilis DSM 1088 heptaprenyl transferase gene are fermented in a fermentation medium, with menaquinone-7 being accumulated in the cells of the fermented E. coli strain.

### BRIEF DESCRIPTION OF THE DRAWINGS

FIG. 1 depicts schematically an operon construct of the invention for menaquinone-7 production, wherein gapA_{P}=E. coli W3110 gapA promoter; RBS=ribosome binding site; hepS=hepS gene from DSM 1088; hepT=hepT gene from DSM 1088; HPT=putative heptaprenyl transferase gene from DSM 1088; (a), (b), (c) and (d)=cloning elements.

FIG. 2 depicts schematically a plasmid, pKG82, which is suitable for producing menaquinone-7.

### DETAILED DESCRIPTION OF PREFERRED EMBODIMENTS

The E. coli strain preferably comprises multiple functional copies of the three genes mentioned. Preferably, it comprises from one to 700 copies of the three genes mentioned. Particularly preferably, it comprises from one to 20 copies of the three genes mentioned. The genes may be on one or several plasmids; they may also be integrated into the chromosome.

The invention therefore likewise comprises a plasmid comprising the B. subtilis DSM 1088 hepS gene, the B. subtilis DSM 1088 hepT gene and a putative B. subtilis DSM 1088 heptaprenyl transferase gene. Such a production plasmid makes possible industrial production of menaquinone-7 in Escherichia coli.

The three genes are defined as follows:

The B. subtilis DSM 1088 hepS gene is preferably characterized by SEQ ID NO 1 and variants of this sequence which are due to the degeneracy of the genetic code or which code for a protein having the function of a HepS subunit in a heptaprenyl synthase. Particular preference is given to said gene being SEQ ID NO 1 and variants of this sequence due to the degeneracy of the genetic code.

The B. subtilis DSM 1088 hepT gene is preferably characterized by SEQ ID NO 2 and variants of this sequence which are due to the degeneracy of the genetic code or which code for a protein having the function of a HepT subunit in a heptaprenyl synthase. Particular preference is given to said gene being SEQ ID NO 2 and variants of this sequence due to the degeneracy of the genetic code.

Preferably, a protein has the function of a HepS subunit in a heptaprenyl synthase if it forms, together with a HepT protein encoded by a hepT gene having SEQ ID NO 2, a heterodimer which has a heptaprenyl synthase activity which corresponds to at least the heptaprenyl synthase activity of a heterodimer of HepS encoded by a gene having SEQ ID NO 1 and HepT encoded by a gene having SEQ ID NO 2.

Preferably, a protein has the function of a HepT subunit in a heptaprenyl synthase if it forms, together with a HepS protein encoded by the hepS gene having SEQ ID NO 1, a heterodimer which has a heptaprenyl synthase activity which corresponds to at least the heptaprenyl synthase activity of a heterodimer of HepT encoded by a gene having SEQ ID NO 2 and HepS encoded by a gene having SEQ ID NO 1.

The putative B. subtilis DSM 1088 heptaprenyl transferase gene is characterized by SEQ ID NO 3 and variants of this sequence which are due to the degeneracy of the genetic code or which code for a protein having heptaprenyl transferase activity. Particular preference is given to said gene being SEQ ID NO 3 and variants of this sequence due to the degeneracy of the genetic code.

The functions of the heptaprenyl synthases and transferases of the invention in E. coli may be characterized indirectly via the product, menaquinone-7, since Escherichia coli wild-type strains do not produce any menaquinone-7. A detection method which may be used is the HPLC method described in example 5.

Production of menaquinone-7 preferably requires expression of the B. subtilis DSM 1088 hepS gene, the B. subtilis DSM 1088 hepT gene and the putative B. subtilis DSM 1088 heptaprenyl transferase gene on a plasmid in E. coli.

Expression of these genes is preferably achieved through homologous or heterologous promoters. The term heterologous relates to the genes characterized by SEQ ID NO 1, SEQ ID NO 2, and SEQ ID NO 3. Examples of appropriate heterologous promoters are the promoter of the E. coli gapA gene or the promoter of the E. coli tufB gene. Other heterologous promoters known to the skilled worker are the lac, tac, trc, lambda, ara and tet promoters.

Preference is given to expression of the B. subtilis DSM 1088 hepS gene, the B. subtilis DSM 1088 hepT gene and the putative B. subtilis DSM 1088 heptaprenyl transferase gene being achieved through the promoter of the E. coli gapA gene.

The plasmid of the invention therefore preferably comprises the above-mentioned promoters which are located on the plasmid in such a way that they direct expression of the B. subtilis DSM 1088 hepS gene, the B. subtilis DSM 1088 hepT gene and the putative B. subtilis DSM 1088 heptaprenyl transferase gene.

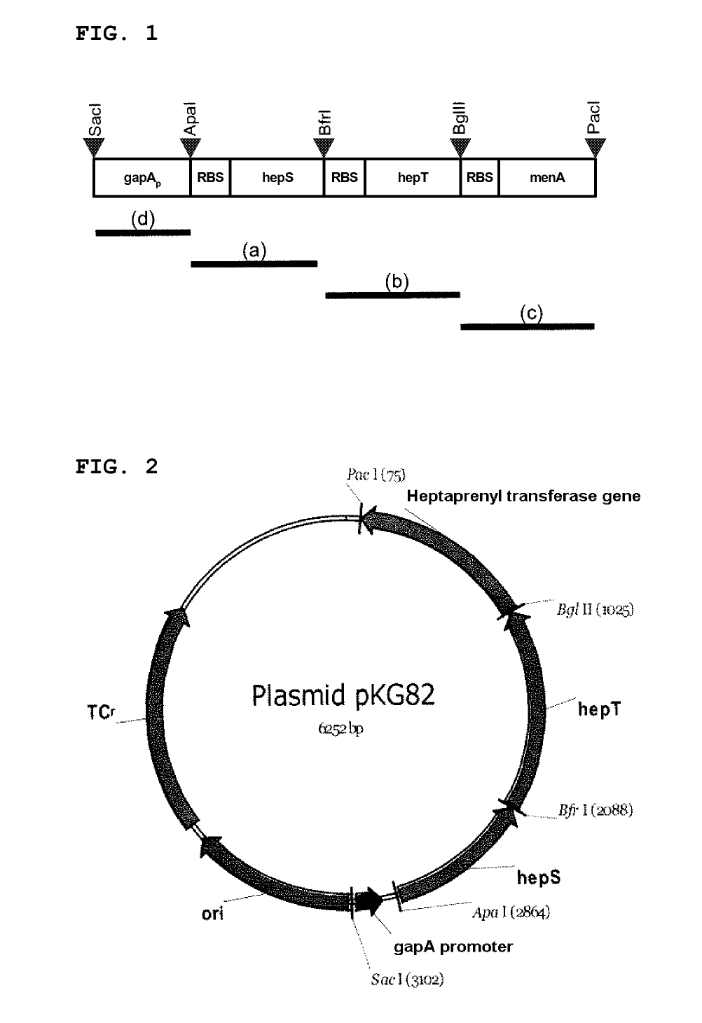

Particular preference is given to the plasmid comprising an operon construction in which the B. subtilis DSM 1088 hepS gene, the B. subtilis DSM 1088 hepT gene and the putative B. subtilis DSM 1088 heptaprenyl transferase gene are under control of the E. coli gapA promoter. An example of such a construct is depicted in FIG. 1.

However, it is also possible for the natural (homologous) promoter regions of these genes to be used as promoter region for expressing the B. subtilis DSM 1088 hepS gene, the B. subtilis DSM 1088 hepT gene and the putative B. subtilis DSM 1088 heptaprenyl transferase gene.

Plasmids incorporating the B. subtilis DSM 1088 hepS gene, the B. subtilis DSM 1088 hepT gene and the putative B. subtilis DSM 1088 heptaprenyl transferase gene that may be used are any available DNA molecules which are accessible to genetic engineering and which are replicated extrachromosomally in Escherichia coli and which comprise a selection marker. Thus it is possible to employ for example plasmids having a high cellular copy number in E. coli (e.g. plasmids of the pUC series, plasmids of the pQE series, plasmids of the pBluescript series), plasmids having an average copy number in E. coli (e.g. plasmids of the pBR series, plasmids of the pACYC series) or plasmids having a low copy number in E. coli (e.g. pSC101 or pBeloBAC11).

Preference is given to using plasmids having an average copy number in E. coli (e.g. plasmids of the pBR series, plasmids of the pACYC series).

Particular preference is given to using a plasmid of the pACYC series.

To produce menaquinone-7, a plasmid of the invention is introduced into an Escherichia coli strain.

This is accomplished, for example, by a common transformation method such as, for example, electroporation or the CaCl₂ method. Plasmid-carrying clones are subsequently selected by antibiotic resistance. Examples of selection markers known to the skilled worker are the chloramphenicol acetyltransferase gene, which imparts resistance to chloramphenicol, the neomycin phosphotransferase gene, which imparts resistance to kanamycin, the tetracycline efflux gene, which imparts resistance to tetracycline, and the β-lactamase gene, which imparts resistance to ampicillin and carbenicillin.

As an alternative to expression from a plasmid of the invention, the B. subtilis DSM 1088 hepS gene, the B. subtilis DSM 1088 hepT gene and the putative B. subtilis DSM 1088 heptaprenyl transferase gene may also be integrated into the chromosome of an E. coli strain, either additionally to or as a replacement for the menaquinone biosynthesis genes already present. Integration methods which are preferably utilized are the systems known to the skilled worker comprising temperate bacteriophages, integrative plasmids or integration by way of homologous recombination.

The invention therefore also relates to E. coli strains which comprise a plasmid of the invention or multiple, preferably in each case from one to five, chromosomal copies of the B. subtilis DSM 1088 hepS gene, the B. subtilis DSM 1088 hepT gene and the putative B. subtilis DSM 1088 heptaprenyl transferase gene.

Menaquinone-7 is produced with the aid of an E. coli strain of the invention in a fermenter by methods known per se.

The E. coli strain is grown in the fermenter by way of a continuous culture, batch culture or, preferably, fed-batch culture.

Preferred carbon sources used are sugars, sugar alcohols or organic acids. Particular preference is given to the method of the invention employing glucose, lactose, sucrose or glycerol as carbon sources.

Preference is given to metering in the carbon source in a way that ensures that the carbon source content in the fermenter is kept within a range from 0.1 g/l to 50 g/l during fermentation. Particular preference is given to a range from 0.1 g/l to 10 g/l.

Preference is given to the method of the invention using ammonia, ammonium salts or protein hydrolysates as the nitrogen source. If ammonia is used as correctant for pH control, this nitrogen source will be continued to be metered in regularly during fermentation.

Other media supplements which may be added are salts of the elements phosphorus, chlorine, sodium, magnesium, nitrogen, potassium, calcium, iron and, in traces (i.e. in μM concentrations), salts of the elements molybdenum, boron, cobalt, manganese, zinc, copper and nickel.

Furthermore, it is possible to add organic acids (e.g. acetate, citrate), amino acids (e.g. L-isoleucine, D/L-methionine) and vitamins (e.g. vitamin B1, vitamin B6, vitamin B12) to the medium.

Examples of complex nutrient sources which may be employed are yeast extract, corn steep liquor, soya meal or malt extract.

The incubation temperature for Escherichia coli is preferably 28-37° C., with particular preference being given to an incubation temperature of 30-32° C.

The pH of the fermentation medium is preferably in the pH range from 5.0 to 8.5 during fermentation, with particular preference being given to a pH of 7.0.

The E. coli strain is incubated under aerobic, anaerobic or microaerobic culturing conditions over a period of from 16 h to 150 h and within the range of the optimal growth temperature for the particular strain. Particular preference is given to culturing times of between 48 h and 96 h.

The fermentation is preferably carried out under aerobic or microaerobic growth conditions.

Menaquinone-7 may be removed from the culture by methods known to the skilled worker, such as centri-fugation of the medium to remove the cells with subsequent extraction of menaquinone-7 from the biomass, chromatographic purification, concentration, formulation or complexing of the product.

Detection and quantification of the menaquinone-7 produced in the method of the invention are carried out, for example, by means of an HPLC method.

### EXAMPLES

The following examples serve to further illustrate the invention.

### Example 1

### Construction of the pKG82 Plasmid

a) Amplification of the hepS Gene:

The Bacillus subtilis (DSM 1088) hepS gene was amplified by means of the polymerase chain reaction (PCR) using the Taq DNA polymerase (Roche, Mannheim, Germany) according to common practice known to the skilled worker. The template used was chromosomal DNA of Bacillus subtilis strain DSM 1088. The primers used were the oligonucleotides M7-Operon-hepS-for (SEQ ID NO 4) and M7-Operon-hepS-rev (SEQ ID NO 5).

The 783 base pair DNA fragment obtained in the PCR was then purified by means of a DNA absorption column of the QIAprep Spin Miniprep Kit (Qiagen, Hilden, Germany) according to the manufacturer's information.

b) Amplification of the hepT Gene:

The Bacillus subtilis (DSM 1088) hepT gene was amplified by means of the polymerase chain reaction (PCR) using the Taq DNA polymerase (Roche, Mannheim, Germany) according to common practice known to the skilled worker. The template used was chromosomal DNA of Bacillus subtilis strain DSM 1088. The primers used were the oligonucleotides M7-Operon-hepT-for (SEQ ID NO 6) and M7-Operon-hepT-rev (SEQ ID NO 7). The 1075 base pair DNA fragment obtained in the PCR was then purified by means of a DNA absorption column of the QIAprep Spin Miniprep Kit (Qiagen, Hilden, Germany) according to the manufacturer's information.

### c) Amplification of the Putative Heptaprenyl Transferase Gene:

The putative Bacillus subtilis (DSM 1088) heptaprenyl transferase gene was amplified by means of the polymerase chain reaction (PCR) using the Taq DNA polymerase (Roche, Mannheim, Germany) according to common practice known to the skilled worker. The template used was chromosomal DNA of Bacillus subtilis strain DSM 1088. The primers used were the oligonucleotides M7-Operon-pHPTG-for (SEQ ID NO 8) and M7-Operon-pHPTG-rev (SEQ ID NO 9).

The 966 base pair DNA fragment obtained in the PCR was then purified by means of a DNA absorption column of the QIAprep Spin Miniprep Kit (Qiagen, Hilden, Germany) according to the manufacturer's information.

d) Amplification of the E. coli gapA Promoter:

The gapA promoter from E. coli W3110 (ATCC 27325) was amplified by means of the polymerase chain reaction (PCR) using the Taq DNA polymerase (Roche, Mannheim, Germany) according to common practice known to the skilled worker. The template used was chromosomal DNA of E. coli strain W3110 (ATCC 27325). The primers used were the oligonucleotides M7-Operon-gapA-for (SEQ ID NO 10) and M7-Operon-gapA-rev (SEQ ID NO 11).

The 319 base pair DNA fragment obtained in the PCR was then purified by means of a DNA absorption column of the QIAprep Spin Miniprep Kit (Qiagen, Hilden, Germany) according to the manufacturer's information.

e) Cloning of the genes hepS, hepT and of the putative heptaprenyl transferase gene into the pACYC184-LH plasmid vector under the control of the gapA promoter:

The DNA fragments from a), b), c) and d) were cut with the following restriction endonucleases (Roche, Mannheim) prior to the ligation reaction with T4 DNA ligase (Roche, Mannheim):

(I): Fragment from a) with Sad and ApaI
(II): Fragment from b) with ApaI and BfrI
(III): Fragment from c) with BfrI and BglII
(IV): Fragment from d) BglII and Pad (New England Biolabs, Frankfurt am Main, Germany).

The pACYC184-LH (DSM 10172) cloning vector was cut with the restriction endonucleases Sad (Roche, Mannheim) and Pad (New England Biolabs, Frankfurt am Main) and then ligated with the cut fragments of (I), (II), (III) and (IV) in a ligation reaction after purification (see FIG. 1). The resulting plasmid was checked for its correct identity by Sanger sequencing. The finished menaquinone-7 production plasmid was referred to as pKG82 (see FIG. 2).

The pKG82 plasmid used for producing menaquinone-7 was deposited with the DSMZ (Deutsche Sammlung für Mikroorganismen and Zellkulturen GmbH [German Collection of Microorganisms and Cell Cultures], D-38142 Braunschweig) under DSM 23159 in accordance with the Budapest Treaty on 27 Nov. 2009.

### Example 2

### Preparation of a Menaquinone-7 Producer Strain

The pKG82 plasmid described in Example 1 was used for transformation of E. coli strain W3110 (ATCC 27325) by means of the CaCl₂ method. After selection on LB agar plates containing 20 mg/l of tetracycline, the plasmid was re-isolated from one of the transformants, cleaved with restriction endonucleases and checked. This strain is referred to as W3110/pKG82 and is suitable for menaquinone-7 production.

### Example 3

### First Preculture (Day Culture) of a Menaquinone-7 Producer Strain for Fermentation

20 ml of LB medium containing glucose (10 g/l tryptone, 5 g/l yeast extract, 5 g/l NaCl, 15 g/l glucose; autoclaved) were admixed with tetracycline×HCl in a sterile 100 ml conical flask (final tetracycline×HCl concentration: 15 mg/l; tetracycline×HCl stock solution: 10 mg/ml in 50% ethanol, sterile-filtered). Producer strain cells were removed from the agar plate using an inoculation loop until at most the culture started to appear slightly cloudy. The precultures were cultured at 32° C. (strains W3110 and W3110/pKG82) and 30° C. (DSM 1088 strain), respectively, and 150 rpm for 8 h.

### Example 4

### Second Preculture (Overnight Culture) of a Menaquinone-7 Producer Strain for Fermentation

The second preculture (overnight culture) was done in a 2 l fermenter with the aid of a Biostat® B DCU apparatus from B. Braun Biotech International (Melsungen, Germany). 20 ml of LB preculture from Example 3 were used for inoculating the 980 ml of production medium consisting of 25 g/l glucose, 0.015 g/l thiamine×HCl, 0.015 g/l tetracycline×HCl, 3 g/l (NH₄)₂SO₄, 0.25 g/l NaCl, 0.9 g/l L-isoleucine, 0.6 g/l D/L-methionine, 30 g/l cornsteep liquor (Roquette, Lestrem, France), 0.03 g/l CaCl₂×2H₂O, 0.6 g/l MgSO₄×7 H₂O, 0.15 g/l Na₂MO₄×2H₂O, 2.5 g/l H₃BO₃, 0.7 g/l CoCl₂×6H₂O, 0.25 g/l CuSO₄×5 H₂O, 1.6 g/l MnCl₂×4H₂O, 0.3 g/l ZnSO₄×7 H₂O, 0.15 g/l FeSO₄×7H₂O, 1 g/l Na₃ citrate×2H₂O and 1.7 g/l KH₂PO₄. The media components were introduced first into a 2 l fermenter in a sterile manner. During the 17 h of cultivation, the temperature was held constant at 32° C. (strains W3110 and W3110/pKG82) or 30° C. (DSM 1088 strain), and the pH was kept constant at pH 7.0 by the correctant (25% ammonia). Struktol J673 (Schill+Seilacher, Hamburg, Germany) in a 1:6 dilution in sterile water was used as antifoam. The culture was gassed with sterile pressurized air at 5 vol/vol/min and stirred with a stirrer at 400 rpm. After oxygen saturation had decreased to 50%, the revolutions were increased to 1500 rpm via the control unit of the fermenter in order to maintain 50% oxygen saturation. Oxygen saturation was determined using a pO₂ probe calibrated to 100% saturation at 400 rpm. As soon as the glucose content in the fermenter had fallen to approx. 5 g/l, a 56% (w/v) glucose solution was metered in. Glucose was fed at a flow rate of 6-12 ml/h, with glucose concentration in the fermenter being held constant between 0.1 g/l and 10 g/l. Glucose was measured using the YSI 7100 MBS analyser (YSI, Yellow Springs, Ohio, USA).

### Example 5

### Fermentative Production of Menaquinone-7

Menaquinone-7 was produced in a fermenter with the aid of a Biostat® B DCU apparatus from B. Braun Biotech International (Melsungen, Germany).

The main fermenter was inoculated with 100 ml of culture from an overnight culture (see Example 4). The fermentation conditions corresponded to those of the pre-fermenter of Example 4. The fermentation time was 96 h. Menaquinone-7 was determined as described in Suvarna et al. (2008, Journal of Bacteriology 180, No. 10, pages 2782-2787). Samples were taken after 24 h, 48 h, 72 h and 96 h.

The quinones were analyzed on an HPLC HP 1200 from Agilent (Böblingen, Germany). The separation column used was a Luna C18(2) RP-HPLC column 5μ (100 mm×4.6 mm) from Phenomenex (Aschaffenburg, Germany). The samples were eluted isocratically with isopropanol/acetonitrile (1:3[ vol/vol]) at a temperature of 40° C. and a flowrate of 0.7 ml/min. The quinones were detected using a UV detector at wavelengths of 207 nm and 280 nm.

Table 1 depicts the menaquinone-7 yields obtained with the various strains.

TABLE 1

Menaquinone-7 [mg/g BTM]

| Strain | 24 h | 48 h | 72 h | 96 h |
|---|---|---|---|---|
| Bacillus subtilis DSM | 0.07 | 0.08 | 0.22 | —^{[2]} |
| 1088^{[1]} | | | | |
| Escherichia coli W3110 | —^{[3]} | —^{[3]} | —^{[3]} | —^{[3]} |
| W3110/pKG82 | 0.21 | 0.84 | 1.19 | 1.59 |

BTM = dry biomass
^{[1]}= owing to the extreme foaming of the strain, the airflow had to be limited to 1 vol/vol/min and the stirrer revolutions had to be limited to 400 rpm after 8 h of cultivation
^{[2]}= fermentation stopped after 72 h
^{[3]}= not detectable

What is claimed is:

1. A method of producing menaquinone-7, comprising fermenting in a fermentation medium cells of an E. coli strain comprising a B. subtilis DSM 1088 hepS gene, a B. subtilis DSM 1088 hepT gene and a putative B. subtilis DSM 1088 heptaprenyl transferase gene, such that menaquinone-7 is accumulated in the cells of the fermented E. coli strain.

2. A menaquinone-7 production plasmid for industrial production of menaquinone-7 using Escherichia coli, said plasmid comprising a B. subtilis DSM 1088 hepS gene, a B. subtilis DSM 1088 hepT gene and a putative B. subtilis DSM 1088 heptaprenyl transferase gene.

3. The menaquinone-7 production plasmid according to claim 2, further comprising a homologous or a heterologous promoter.

4. The menaquinone-7 production plasmid according to claim 3, wherein the heterologous promoter is a promoter of an E. coli gapA gene or a promoter of an E. coli tufB gene, or a lac, tac, trc, lambda, ara or tet promoter.

5. The menaquinone-7 production plasmid according to claim 2, further comprising an operon construct in which the B. subtilis DSM 1088 hepS gene, the B. subtilis DSM 1088 hepT gene and the putative B. subtilis DSM 1088 heptaprenyl transferase gene are under the control of an E. coli gapA promoter.

6. The menaquinone-7 production plasmid according to claim 2, wherein the plasmid used is a DNA molecule which can be replicated extrachromosomally in Escherichia coli and which comprises a selection marker.

7. The menaquinone-7 production plasmid according to claim 6, wherein a plasmid having a high cellular copy number in E. coli or a plasmid having an average copy number in E. coli or a plasmid having a low copy number in E. coli is used.

8. An E. coli strain comprising a plasmid according to claim 2 or multiple chromosomal copies of the B. subtilis DSM 1088 hepS gene, the B. subtilis DSM 1088 hepT gene and the putative B. subtilis DSM 1088 heptaprenyl transferase gene.

9. The method according to claim 1, wherein the fermenting is followed by removing menaquinone-7 by centrifugation of the fermentation medium to remove the cells and subsequent extraction of menaquinone-7 from said cells, chromatographic purification, concentration, formulation or complexing of menaquinone-7.
